(19) United States
(12) Patent Application Publication
(10) Pub. No.: US 2019/0083162 A1
(43) Pub. Date: Mar. 21, 2019

(54) ELECTRODE DISCONNECT DETECTION

(71) Applicant: Biosense Webster (Israel) Ltd., Yokneam (IL)

(72) Inventors: Mikhael Feldchtein, Yokneam (IL); Ella Ozeri, Yokneam (IL); Yaron Ephrath, Karkur (IL)

(21) Appl. No.: 15/708,915

(22) Filed: Sep. 19, 2017

(51) Int. Cl. A61B 18/12; A61B 18/16

(52) U.S. Cl. CPC A61B 18/1233; A61B 2018/00577; A61B 2018/124; A61B 18/16

(57) ABSTRACT

An apparatus includes at least one electrode configured to contact a body tissue of a living subject, and an electrosurgical generator, which is configured to drive radio-frequency electrical currents through the at least one electrode and to measure the electrical currents passing through the at least one electrode. The apparatus includes a neutral electrode configured for attachment to a body surface of the living subject. The apparatus also includes a switch connected between the neutral electrode and the generator, and a controller coupled to open and close the switch and to assess, based on a response of the measured electrical currents to opening and closing of the switch, whether the neutral electrode is attached to the body surface of the subject.

### FIELD OF THE INVENTION

The present invention relates generally to electrosurgery, and particularly to an apparatus for detecting the integrity of the electrosurgery circuitry.

### BACKGROUND

In an electrosurgical procedure a neutral electrode is attached to the subject and acts as a return electrode for currents generated for the electrosurgery.

U.S. Pat. No. 5,087,257 describes neutral electrodes insulated from each other. The neutral electrodes are separately connected to the current return terminal of a high frequency electro-surgical equipment, and are in electrical contact with the skin of a subject while surgery is performed with the active electrode of the equipment.

U.S. Pat. No. 5,246,439 describes electrosurgery equipment which has an alarm circuit for detecting when a return plate electrode is incorrectly attached.

U.S. Pat. No. 6,007,532 describes a method of monitoring the contact of a biomedical electrode to skin of a subject, where at least two different frequencies are employed to periodically monitor total contact impedance of the electrode.

U.S. Pat. No. 7,160,293 describes a multiple RF return pad contact detection system, which is adaptive to different physiological characteristics of subjects without being susceptible to electrosurgical current interference (e.g., interference or measurement interaction between components of the detection system).

U.S. Pat. 4,171,700 describes a high-frequency surgical apparatus having a number of electrode-connections for a neutral electrode and a number of electrodes, respectively, for monopolar and bipolar operation. In the apparatus two high-frequency generators are connected to a single voltage source, so that the second generator may serve exclusively for supplying a high-frequency current to the electrode-connections for bipolar electrodes.

U.S. Pat. 4,237,887 describes a radio-frequency electrosurgical device which includes an active electrode, a passive electrode, and means for generating a radio-frequency potential.

U.S. Pat. No. 4,754,757 describes a HF-current generator of an electrosurgical apparatus, which has one terminal connected to an active electrode and an additional terminal connected to a neutral electrode.

U.S. Pat. No. 4,848,335 describes an apparatus for monitoring the resistance of the return part of the subject circuit of either a dual foil or single foil return electrode according to a user's selection.

U.S. Pat. No. 7,425,835 describes a method and a measurement apparatus for determining the transition impedance between two electrode parts of a subdivided neutral electrode used in high-frequency surgery.

U.S. Pat. No. 7,637,907 describes a system for monitoring a plurality of return electrodes.

U.S. Pat. No. 8,449,536 describes neutral electrodes that include, in addition to known components, at least one measuring electrode spaced from the main electrode, and at least one measuring current generator that is connected to the main electrode and to the measuring electrode generating a high frequency measuring current, which flows between the measuring electrode and the main electrode.

U.S. Pat. No. 8,628,524 describes a return electrode detection and monitoring system and method.

U.S. Pat. No. 8,790,337 describes a return electrode monitoring (“REM”) system.

U.S. Patent Application 20130138097 describes a medical device, including an ablation system with a generator, at least one ablation element, a subject return electrode, and a feedback system to verify and monitor the electrical connection between the generator and the subject return electrode, as well as contact of the subject return electrode with the subject.

U.S. Patent Application 20140249523 describes systems, apparatus, and methods for monitoring electrosurgical systems.

U.S. Patent Application 20140350546 describes a system for determining probability of tissue damage. The system includes a plurality of return electrodes adhered to a subject and adapted to couple to an electrosurgical generator configured to generate an electrosurgical current.

### SUMMARY

Embodiments of the present invention that are described hereinbelow provide for an improved apparatus for detecting a disconnect of a neutral electrode in an electrosurgical circuitry.

There is therefore provided, in accordance with an embodiment of the present invention, an apparatus that includes at least one electrode configured to contact a body tissue of a living subject, an electrosurgical generator, which is configured to drive radio-frequency electrical currents through the at least one electrode and to measure the electrical currents passing through the at least one electrode, a neutral electrode which is configured for attachment to a body surface of the living subject, a switch connected between the neutral electrode and the generator, and a controller coupled to open and close the switch and to assess, based on a response of the measured electrical currents to opening and closing of the switch, whether the neutral electrode is attached to the body surface of the subject.

In an embodiment the electrosurgical generator is configured to generate an alternative electrical current for ablating the body tissue.

In another embodiment the controller, upon assessing that the neutral electrode is not attached to the body surface of the subject, is configured to raise an alarm.

There is further provided, in accordance with an embodiment of the present invention, an apparatus that includes at least one electrode configured to contact a body tissue of a living subject, an electrosurgical generator, which is configured to drive radio-frequency electrical currents through the at least one electrode and to measure voltages at the at least one electrode, a neutral electrode configured for attachment to a body surface of the living subject, a switch connected between the neutral electrode and the generator, and a controller coupled to open and close the switch and to assess, based on a response of the measured voltages to opening and closing of the switch, whether the neutral electrode is attached to the body surface of the subject.

In a disclosed embodiment the electrosurgical generator is configured to generate an alternative electrical current for ablating the body tissue.

In another disclosed embodiment the controller, upon assessing that the neutral electrode is not attached to the body surface of the subject, is configured to raise an alarm.

There is also provided, in accordance with an embodiment of the present invention, a method, including inserting at least one electrode into a lumen of a living subject so as to contact a body tissue of the living subject, driving from an electrosurgical generator radio-frequency electrical currents through the at least one electrode and measuring the electrical currents passing through the at least one electrode, attaching a neutral electrode to a body surface of the living subject, connecting a switch between the neutral electrode and the electrosurgical generator, and opening and closing the switch while measuring the electrical currents through the at least one electrode, and assessing, based on a response of the measured electrical currents to the opening and closing of the switch, whether the neutral electrode is attached to the body surface of the subject.

In another embodiment the method includes configuring the electrosurgical generator to generate an alternative electrical current for ablating the body tissue.

In yet another embodiment the method includes configuring the controller to raise an alarm upon the controller assessing that the neutral electrode is not attached to the body surface of the subject.

There is further provided a method including inserting at least one electrode into a lumen of a living subject so as to contact a body tissue of the living subject, driving from an electrosurgical generator radio-frequency electrical currents through the at least one electrode and measuring voltages at the at least one electrode, attaching a neutral electrode to a body surface of the living subject, connecting a switch between the neutral electrode and the electrosurgical generator, and opening and closing the switch, while measuring the voltages, and assessing, based on a response of the measured voltages to the opening and closing of the switch, whether the neutral electrode is attached to the body surface of the subject.

In another embodiment the method includes configuring the electrosurgical generator to generate an alternative electrical current for ablating the body tissue.

In yet another embodiment the method includes configuring the controller to raise an alarm upon the controller assessing that the neutral electrode is not attached to the body surface of the subject.

The present invention will be more fully understood from the following detailed description of the embodiments thereof, taken together with the drawings in which:

### BRIEF DESCRIPTION OF THE DRAWINGS

FIG. 1 is a pictorial illustration of an electrosurgical apparatus, in accordance with a disclosed embodiment of the invention;

FIG. 2 is a schematic illustration of an electrosurgical generator connected to electrodes of a catheter with a neutral electrode in contact with a body surface of a subject, in accordance with a disclosed embodiment of the invention;

FIG. 3 is a schematic illustration of the electrosurgical generator connected to electrodes of the catheter with the neutral electrode disconnected from the body surface of the subject, in accordance with a disclosed embodiment of the invention; and

FIG. 4 is a flowchart of steps of an integrity test of the neutral electrode, in accordance with a disclosed embodiment of the invention.

### DETAILED DESCRIPTION OF EMBODIMENTS

In an electrosurgical procedure an electrosurgical generator injects an ablation current into one or more electrodes of a catheter inserted in a lumen inside a subject's body. The electrosurgical generator is typically a multi-channel radio-frequency (RF) generator, which may typically function as a current source or a voltage source. A neutral electrode is attached to the subject's body surface (skin) and acts as a return electrode to the electrosurgical generator for the ablation currents. In order to prevent skin burns, the neutral electrode has a large surface area and consequently the current through this electrode has a low current density. It is important to detect any disconnect of the neutral electrode from the subject, as—in the case of such a disconnect—the ablation current could return via alternative conductive routes. Ablation currents flowing through these alternative routes may cause tissue burns, and—in case these routes include any of the electrodes used for ablation—even undesired, highly dangerous ablation. For these reasons, standards for medical equipment require a safe connection of the neutral electrode.

The electrosurgical generator is controlled by a physician assisted by a computerized controller. The tissue of the subject connecting the catheter electrodes to each other and to the neutral electrode forms a continuous, distributed impedance network. During the electrosurgical procedure the controller monitors and displays, for the purposes of ablation control, the RF power flowing through each of the active electrodes (as opposed to electrodes that may not be energized) into this impedance network. The monitored power levels may remain within acceptable values even when the neutral electrode is disconnected, and are thus not a sufficient monitor for a loss of integrity (disconnect) of the neutral electrode. Presently, for a split neutral electrode, the relevant standard provides that the generator monitors the integrity of the connection of the neutral electrode to the subject's skin. For a solid neutral electrode the staff attending to the subject and to the electrosurgical equipment monitor this integrity visually.

The embodiments of the present invention that are described herein address the above limitations so as to enable automatic and efficient monitoring of the integrity of the connection of the neutral electrode to the subject's skin.

In an embodiment of the present invention, at least one electrode is configured to contact a body tissue of a living subject. An electrosurgical generator is configured to drive radio-frequency electrical currents through the at least one electrode and to measure the electrical currents passing through the at least one electrode, and voltages on tissue contacting the at least one electrode. A neutral electrode is configured for attachment to a body surface of the living subject, and a switch is connected between the neutral electrode and the electrosurgical generator. A controller is coupled to open and close the switch and to assess, based on a response of the measured currents and/or voltages to opening and closing of the switch, whether the neutral electrode is attached to the body surface of the subject.

The controller is configured to raise an alarm upon assessing that the neutral electrode is not attached to the body of the subject.

Typically, the electrosurgical generator is configured as a current source for ablating the body tissue.

Although the above embodiments describe monitoring the integrity of the neutral electrode contact, they may also be utilized for monitoring the integrity of the contact of other electrodes of the electrosurgical apparatus, such as the active body surface electrodes.

### System Description

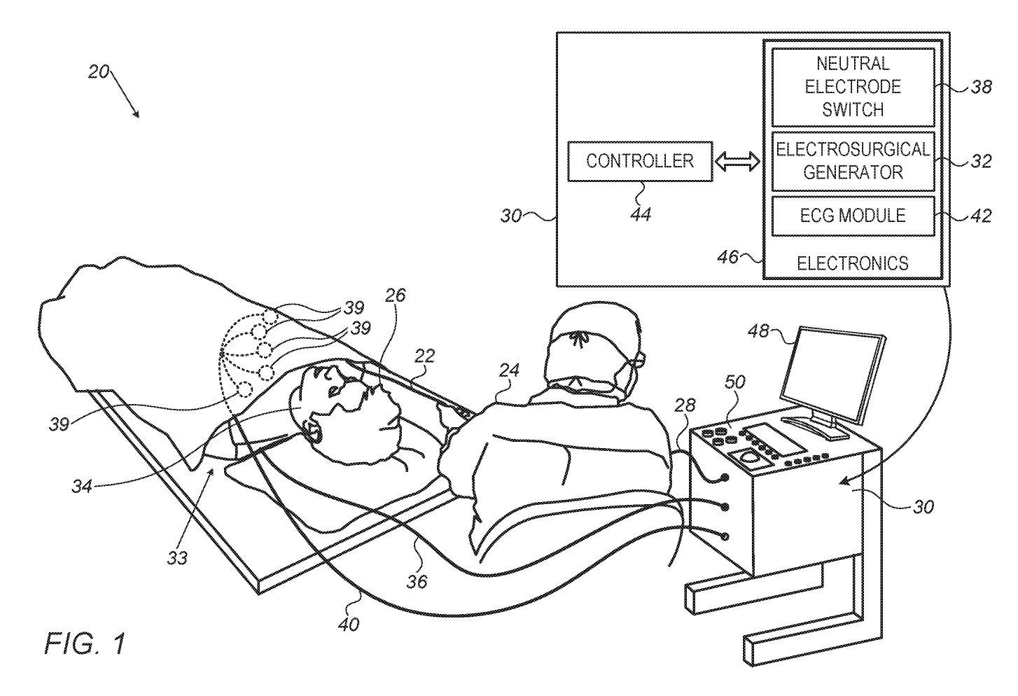

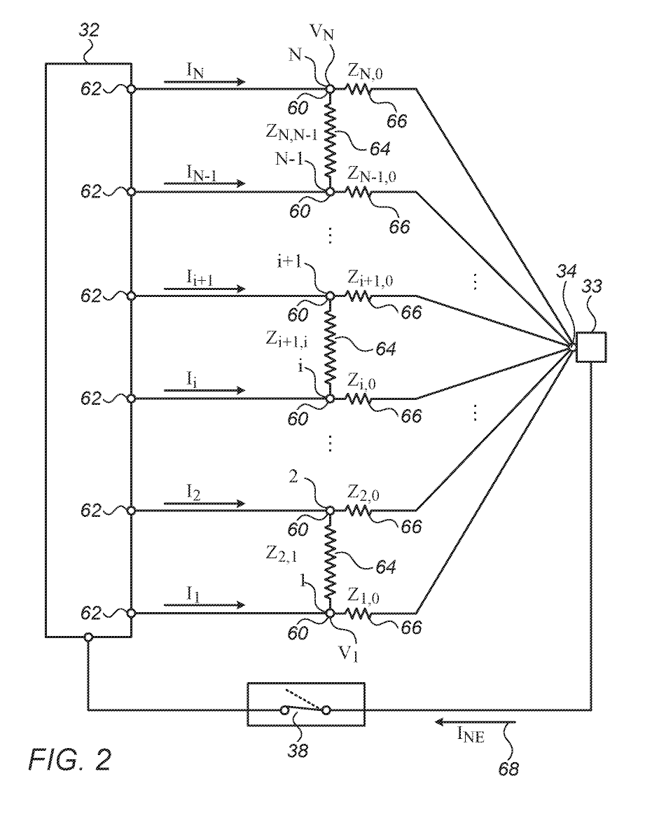

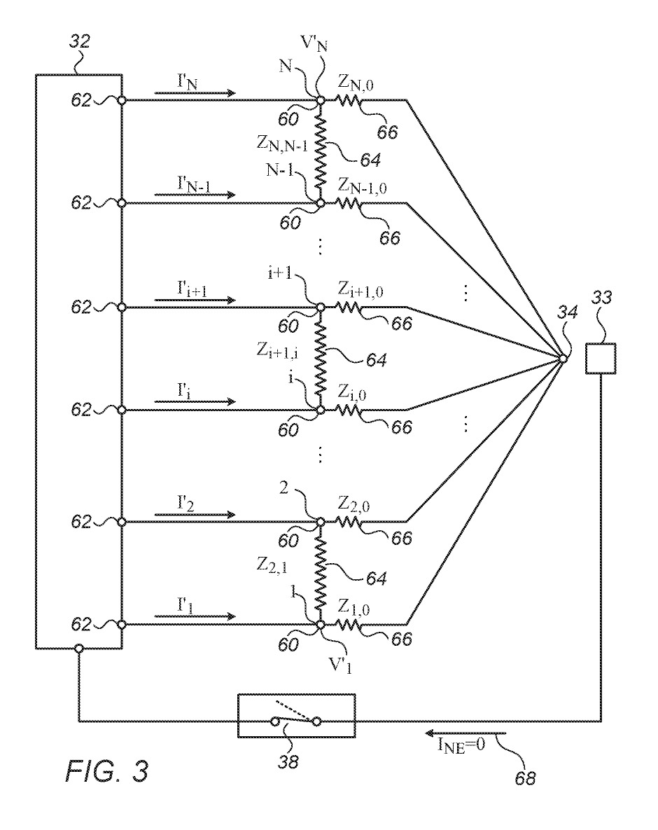

FIG. 1 is a pictorial illustration of an electrosurgical apparatus 20, in accordance with a disclosed embodiment of the invention. Apparatus 20 typically comprises an electrosurgery catheter 22, which is percutaneously inserted by an operator 24, who is typically a physician, through a vascular system of a subject 26 into a lumen (not shown) inside the subject. At a distal end of catheter 22 are located several electrodes 60 (also shown schematically in FIGS. 2-3) in contact with the body tissue of subject 26 for the purpose of ablating the tissue with RF current. A proximal end 28 of catheter 22 is connected to a console 30 and further to an electrosurgical generator 32.

A neutral electrode 33 (typically attached to the back of subject 26, and shown schematically in FIGS. 2-3) is attached to a body surface (skin) 34 of subject 26 for providing a return path for the ablation currents injected by electrosurgical generator 32 into electrodes 60 on catheter 22. Neutral electrode 33 is connected by a cable 36 to console 30 and further to electrosurgical generator 32 via a neutral electrode switch 38.

Body surface ECG electrodes 39 are attached to body surface 34 of subject 26 for providing electrophysiological signals from the subject. ECG electrodes 39 are connected via a cable 40 to console 30 and further to an ECG module 42. The module receives the signals from electrodes 39, and uses the signals, typically after noise reduction, to inter alia provide operator 24 with graphical and numerical displays of the signals on a display 48.

Apparatus 20 is controlled by a controller 44, which is located in console 30. Controller 44 communicates with electronics 46, which comprises electrosurgical generator 32, neutral electrode switch 38, ECG module 42, and additional modules for operating apparatus 20. Electronics 46 are also located in console 30. Controller 44 may comprise a general purpose or embedded computer controller, which is programmed with suitable software for carrying out the functions described hereinbelow. The software may be provided to controller 44 on tangible non-transitory media, such as CD-ROM (Compact Disc Read-Only Memory) or non-volatile memory. Alternatively or additionally, the apparatus 20 may comprise a digital signal controller or hard-wired logic.

Apparatus 20 typically comprises display 48 and controls 50, connected to console 30, for the use of operator 24.

FIG. 2 is a schematic illustration of electrosurgical generator 32 connected to electrodes 60 of catheter 22 and to neutral electrode 33 in contact with body surface 34 of subject 26, in accordance with a disclosed embodiment of the invention. Electrosurgical generator 32 is configured to have N multiple outputs 62 that are respectively connected to N electrodes 60, wherein a typical number N of electrodes is ten. For the sake of clarity, N electrodes 60 are labelled in FIG. 2 by consecutive positive integers 1, 2, . . . N.

A current from any electrode 60 to any other electrode 60 and to neutral electrode 33 takes any available path through the tissue of subject 26. Consequently, the impedances connecting electrodes 60 and neutral electrode 33 are distributed in the tissue. For the sake of clarity and simplicity, these impedances are hereinbelow described as discrete impedances 64 and 66, and the embodiments of the present invention are described using these discrete impedances, without any loss of accuracy or generality, so that embodiments of the present invention comprise distributed impedances.

In the discrete representation, impedances 64 comprise the impedances between neighboring electrodes 60, and are labelled by Z_{i+1,i} for an impedance between electrodes i+1 and i. Impedances 66 comprise the impedances from each electrode 60 to neutral electrode 33, and are labelled by Z_{i,0} for an impedance from electrode i to the neutral electrode. i is an integer from 1 to N.

During a typical electrosurgical ablation procedure, electrosurgical generator 32 injects RF currents I_{I} . . . I_{N} into electrodes 60, with an accompanying voltage V_{N} at each electrode.

Periods between ablations are herein called idle periods. During these idle periods, electrosurgical generator 32 sends low-level RF currents so that the total of the currents does not exceed 10 mA, to electrodes 60.

As the RF currents are AC currents (alternating currents), wherein the actual current reverses its direction once every cycle, the expressions indicating direction and flow of current are used for descriptive purposes only.

When neutral electrode 33 is attached to body surface 34 of subject 26, and switch 38, which connects the neutral electrode to electrosurgical generator 32, is in a closed state, a return current I_{NE}, denoted by an arrow 68, balances—according to Kirchhoff's current law—the currents flowing through impedances 66 into the neutral electrode.

At all times, i.e., during an idle period and while ablation surgery is being performed, electrosurgical generator 32 typically functions in one of two modes: a current source mode or a voltage source mode. In the current source mode, electrosurgical generator 32 measures currents I₁ . . . I_{N} flowing from each output 62 to each electrode 60, and voltages Vᵢ . . . V_{N} at each output 62, while keeping currents I₁ . . . I_{N} substantially constant. In the voltage source mode, electrosurgical generator 32 measures currents I₁ . . . I_{N} flowing from each output 62 to each electrode 60, and voltages Vᵢ . . . V_{N} at each output 62, while keeping voltages Vᵢ . . . V_{N} substantially constant.

In a disclosed embodiment, electrosurgical generator 32 sends the measured currents or voltages to controller 44. In an alternative embodiment the electrosurgical generator 32 sends the measured currents and voltages to controller 44. For clarity, in the following description, the electrosurgical generator is assumed to send both the measured currents and voltages to controller 44, and those having ordinary skill in the art will be able to adapt the description, mutatis mutandis, for cases such as the above-referenced disclosed embodiment, where the generator sends either measured currents or measured voltages to the controller.

Due to the fact that the impedance between each output 62 and corresponding electrode 60 is essentially zero and each output is connected to exactly one electrode, the currents and voltages measured at the outputs are the same as those at the corresponding electrodes.

During the electrosurgical ablation procedure, neutral electrode switch 38 is in a closed state and provides a return path for return current I_{NE}, indicated by arrow 68, flowing from neutral electrode 33 through the neutral electrode switch to electrosurgical generator 32.

The integrity of the contact of neutral electrode 33 to body surface 34 is monitored during the idle period, while electrosurgical generator 32 injects very low currents into electrodes 60. For the purpose of monitoring, controller 44 briefly opens neutral electrode switch 38, thus cutting off return current I_{NE}, while electrosurgical generator 32 continues measuring the currents injected into electrodes 60 and the voltages at these electrodes. Controller 44 keeps receiving the measured currents and voltages from electrosurgical generator 32. If neutral electrode 33 is attached to body surface 34, opening neutral electrode switch 38 changes the impedance from neutral electrode 33 to electrosurgical generator 32 from essentially zero to an infinite impedance.

According the Kirchhoff's current law, the opening of switch 38 changes the current distribution at the point where neutral electrode 33 is attached to body surface 34. In the case where electrosurgical generator 32 functions in current source mode, the opening of switch 38 changes the voltages measured on electrodes 60, and in the case of voltage source mode, this changes the currents measured on electrodes 60. This change, observed by controller 44 responsively to the opening of neutral electrode switch 38, is an indication that neutral electrode 33 is in good contact with body surface 34.

FIG. 3 is a schematic illustration of electrosurgical generator 32 connected to electrodes 60 of catheter 22 with neutral electrode 33 disconnected from body surface 34 of subject 26, in accordance with a disclosed embodiment of the invention. Electrosurgical generator 32 and its connections to electrodes 60 as well as the impedance network comprising impedances 64 and 66 are substantially identical to those depicted in FIG. 2. During an idle period, the currents into electrodes 60 and the voltages at these electrodes are labelled, respectively, as I′₁ . . . I′_{N} and V′₁ . . . V′_{N}, as they typically differ from those of FIG. 2.

A difference between FIG. 2 and FIG. 3 is what is observed by controller 44 when neutral electrode switch 38 is opened and closed: Due to the disconnect of neutral electrode 33 from body surface 34, the current distribution in the impedance network is independent of the state of neutral electrode switch 38. Consequently, the currents I′₁ . . . I′_{N} and voltages V′₁ . . . V′_{N} measured by electrosurgical generator 32 and communicated to controller 44 do not change when neutral electrode switch 38 is toggled between open and closed states. From the lack of response of the respectively measured currents or voltages to the toggling of neutral electrode switch 38, controller 44 concludes that neutral electrode 33 is not attached to body surface 34. Based on this result, controller 44 signals, by raising an alarm, to operator 24 that neutral electrode 33 is disconnected from subject 26, enabling the operator to reconnect the electrode. The alarm may comprise displaying a message on display 48, flashing a light, or sounding a warning signal.

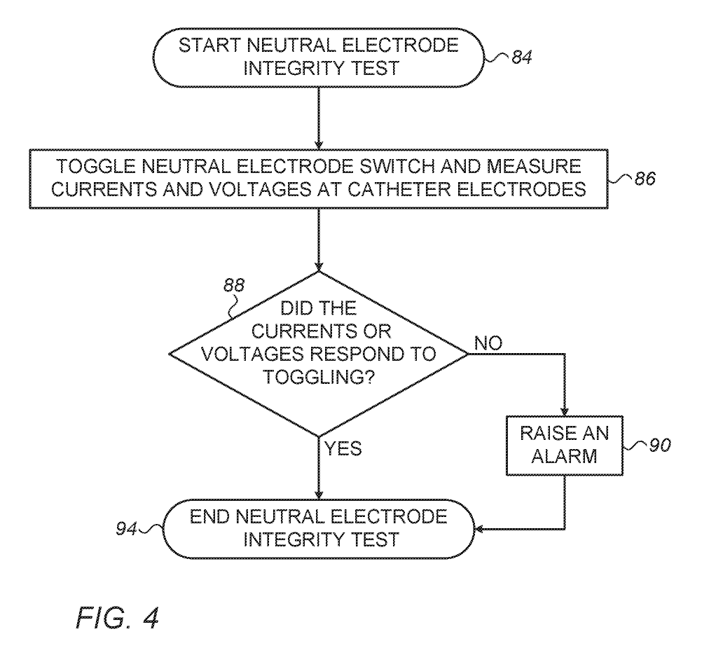

FIG. 4 is a flowchart of the integrity test of neutral electrode 33, in accordance with a disclosed embodiment of the invention. The integrity test takes place during the idle period of electrosurgical apparatus 20, as described in the context of FIG. 2. In a start step 84 the integrity test is started. In a toggle step 86 neutral electrode switch 38 is toggled between open and closed while measuring the currents flowing through electrodes 60 and the voltages at these electrodes, as described in the context of FIG. 2.

In an evaluation step 88, the responses of, respectively, the currents or voltages (measured in toggle step 86) to the toggling of neutral electrode switch 38 are evaluated. If the currents or voltages, respectively, do not respond to the toggling, controller 44 raises an alarm, detailed in the context of FIG. 3, in a disconnect alarm step 90, warning the operator that neutral electrode 33 is disconnected from subject 26, and the integrity test ends in an end step 94. If the currents or voltages respond to the toggling, no further action is taken, and the integrity test ends in end step 94.

It will be appreciated that the embodiments described above are cited by way of example, and that the present invention is not limited to what has been particularly shown and described hereinabove. Rather, the scope of the present invention includes both combinations and subcombinations of the various features described hereinabove, as well as variations and modifications thereof which would occur to persons skilled in the art upon reading the foregoing description and which are not disclosed in the prior art.

1. An apparatus comprising:
at least one electrode configured to contact a body tissue of a living subject;
an electrosurgical generator, which is configured to drive radio-frequency electrical currents through the at least one electrode and to measure the electrical currents passing through the at least one electrode;
a neutral electrode configured for attachment to a body surface of the living subject;
a switch connected between the neutral electrode and the generator; and
a controller coupled to open and close the switch and to assess, based on a response of the measured electrical currents to opening and closing of the switch, whether the neutral electrode is attached to the body surface of the subject.

2. The apparatus according to claim 1, wherein the electrosurgical generator is configured to generate an alternative electrical current for ablating the body tissue.

3. The apparatus according to claim 1, wherein the controller, upon assessing that the neutral electrode is not attached to the body surface of the subject, is configured to raise an alarm.

4. An apparatus comprising:
at least one electrode configured to contact a body tissue of a living subject;
an electrosurgical generator, which is configured to drive radio-frequency electrical currents through the at least one electrode and to measure voltages at the at least one electrode;
a neutral electrode configured for attachment to a body surface of the living subject;
a switch connected between the neutral electrode and the generator; and
a controller coupled to open and close the switch and to assess, based on a response of the measured voltages to opening and closing of the switch, whether the neutral electrode is attached to the body surface of the subject.

5. The apparatus according to claim 4, wherein the electrosurgical generator is configured to generate an alternative electrical current for ablating the body tissue.

6. The apparatus according to claim 4, wherein the controller, upon assessing that the neutral electrode is not attached to the body surface of the subject, is configured to raise an alarm.

7. A method comprising:
inserting at least one electrode into a lumen of a living subject so as to contact a body tissue of the living subject;
driving from an electrosurgical generator radio-frequency electrical currents through the at least one electrode and measuring the electrical currents passing through the at least one electrode;
attaching a neutral electrode to a body surface of the living subject;
connecting a switch between the neutral electrode and the electrosurgical generator; and
opening and closing the switch while measuring the electrical currents through the at least one electrode, and assessing, based on a response of the measured electrical currents to the opening and closing of the switch, whether the neutral electrode is attached to the body surface of the subject.

8. The method according to claim 7, and comprising configuring the electrosurgical generator to generate an alternative electrical current for ablating the body tissue.

9. The method according to claim 7, comprising configuring the controller to raise an alarm upon the controller assessing that the neutral electrode is not attached to the body surface of the subject.

10. A method comprising:
inserting at least one electrode into a lumen of a living subject so as to contact a body tissue of the living subject;
driving from an electrosurgical generator radio-frequency electrical currents through the at least one electrode and measuring voltages at the at least one electrode;
attaching a neutral electrode to a body surface of the living subject;
connecting a switch between the neutral electrode and the electrosurgical generator; and
opening and closing the switch, while measuring the voltages, and assessing, based on a response of the measured voltages to the opening and closing of the switch, whether the neutral electrode is attached to the body surface of the subject.

11. The method according to claim 10, and comprising configuring the electrosurgical generator to generate an alternative electrical current for ablating the body tissue.

12. The method according to claim 10, comprising configuring the controller to raise an alarm upon the controller assessing that the neutral electrode is not attached to the body surface of the subject.
